(19) United States
(12) Patent Application Publication
(10) Pub. No.: US 2024/0424324 A1
(43) Pub. Date: Dec. 26, 2024

(54) QUANTUM PRODUCTION AND SUPPLY OF ONE OR MORE BREATHING OR OTHER SUBSTANCES TO AN ORGANISM

(71) Applicant: Addison M. FISCHER, Naples, FL (US)

(72) Inventor: Addison M. FISCHER, Naples, FL (US)

(21) Appl. No.: 18/396,433

(22) Filed: Dec. 26, 2023

Related U.S. Application Data
(63) Continuation of application No. 18/144,794, filed on May 8, 2023, now abandoned.
Application No. 18/144,794 is a continuation of application No. 17/950,543, filed on Sep. 22, 2022, now abandoned.
Application No. 17/950,543 is a continuation of application No. 17/672,193, filed on Feb. 15, 2022, now abandoned.
Application No. 17/672,193 is a continuation of application No. 17/320,816, filed on May 14, 2021, now abandoned.
Application No. 17/320,816 is a continuation of application No. 17/141,875, filed on Jan. 5, 2021, now abandoned.
Application No. 17/141,875 is a continuation-in-part of application No. 17/126,588, filed on Dec. 18, 2020, now abandoned.
(60) Provisional application No. 62/957,266, filed on Jan. 5, 2020.
(60) Provisional application No. 62/980,110, filed on Feb. 21, 2020.
(60) Provisional application No. 62/985,834, filed on Mar. 5, 2020.
(60) Provisional application No. 62/980,111, filed on Feb. 21, 2020.
(60) Provisional application No. 62/951,244, filed on Dec. 20, 2019.
(60) Provisional application No. 62/985,840, filed on Mar. 5, 2020.
(60) Provisional application No. 62/980,113, filed on Feb. 21, 2020.
(60) Provisional application No. 62/951,576, filed on Dec. 20, 2019.
(60) Provisional application No. 62/985,838, filed on Mar. 5, 2020.
(60) Provisional application No. 62/950,012, filed on Dec. 18, 2019.
(60) Provisional application No. 62/985,841, filed on Mar. 5, 2020.
(60) Provisional application No. 62/962,658, filed on Jan. 17, 2020.
(60) Provisional application No. 62/988,634, filed on Mar. 12, 2020.
(60) Provisional application No. 63/074,887, filed on Sep. 4, 2020.
(60) Provisional application No. 63/090,664, filed on Oct. 12, 2020.
(60) Provisional application No. 62/985,823, filed on Mar. 5, 2020.

(51) Int. Cl. A62B 7/08; A01G 9/24; A61F 9/00; A61G 10/04; A61M 5/14; A61M 16/00; A61M 16/06; A61M 16/10; A61M 31/00; A61M 35/00; A62B 9/06; A62B 18/02; A62B 21/00; B63C 11/24; B63G 8/36; B64G 1/46

(52) U.S. Cl. CPC A62B 7/08; A61F 9/0008; A61G 10/04; A61M 5/14; A61M 16/022; A61M 16/0672; A61M 16/1005; A61M 31/00; A61M 35/30; A62B 9/06; A62B 18/025; A62B 21/00; B63C 11/24; B63G 8/36; B64G 1/46; A01G 9/24; A61M 2202/0208; A61M 2202/0225; A61M 2202/025; A61M 2202/0266; A61M 2205/8206; A61M 2210/0618; A61M 2210/0625; A61M 2210/1067; A61M 2210/1475

(57) ABSTRACT

Systems and methods of providing one or more chemical substance(s) to an organism such as a human, an animal or a plant. One non-limiting embodiment provides a quantum independent breathing apparatus (“QUIBA”) for using NQED(s) technology to assemble, or instantiate, or “quantum print,” fermions comprising a breathable atmosphere, inviting the prospect of longer forays into an inhospitable atmosphere or environment.

### CROSS-REFERENCE TO RELATED APPLICATIONS

The present application is a continuation of application Ser. No. 18/144,794, filed May 8, 2023, which is a continuation of application Ser. No. 17/950,543, filed Sep. 22, 2022, which is a continuation of application Ser. No. 17/672,193, filed Feb. 15, 2022, which is a continuation of application Ser. No. 17/320,816, filed May 14, 2021; which is a continuation of application Ser. No. 17/141,875 filed Jan. 5, 2021, which claims the benefit of priority from application nos. 62/980,110 filed Feb. 21, 2020 and 62/957,266 filed Jan. 5, 2020, each entitled “Quantum Independent Breathing Apparatus Quiba” and claims the benefit of 62/985,823 filed 2020 Mar. 5; and which is a continuation-in-part of application Ser. No. 17/126,588 filed 2020 Dec. 18, which claims the benefit of the following provisional applications:

| | | |
|---|---|---|
| 62950012 | filed | Dec. 18, 2019 |
| 62951244 | filed | Dec. 20, 2019 |
| 62951576 | filed | Dec. 20, 2019 |
| 62962658 | filed | 2020 Jan. 17 |
| 62980111 | filed | Feb. 21, 2020 |
| 62980113 | filed | Feb. 21, 2020 |
| 62985834 | filed | Mar. 5, 2020 |
| 62985838 | filed | Mar. 5, 2020 |
| 62985840 | filed | Mar. 5, 2020 |
| 62985841 | filed | 2020 Mar. 5 |
| 62988634 | filed | 2020 Mar. 12 |
| 63074887 | filed | 2020 Sep. 4 |
| 63090664 | filed | Oct. 12, 2020 |

This application is related to:
Ser. No. 17/127,469 filed Dec. 18, 2020;
Ser. No. 17,127,551 filed Dec. 18, 2020
Ser. No. 17/126,588 filed Dec. 18, 2020;
Ser. No. 17,128,930 filed Dec. 21, 2020; and
PCT/US2020/065887 filed Dec. 18, 2020 entitled “Fuel And Chemical Feedstock Instantiating Source”.

All of the above patent applications are incorporated herein by reference for all purposes as if expressly set forth herein.

### FIELD

This patent discloses a generalized teaching that provides a framework by which Nagel Quantum Effect Devices (NQEDs) can be used to instantiate, or quantum print, chemical substances for use or consumption by a living organism such as for example a human being, an animal or a plant. The technology herein also relates to using such NQEDs to produce chemical substances, such as reductants chemical feedstocks—as well as using NQEDs to instantiate, or quantum print oxidizers (or oxidant or oxidizing agents) such as oxygen (O₂) and many other oxidizing agents, for application to a living organism. The technology further relates to systems including the above that are coupled to means that process such reductants and oxidizers for application to a living organism. One advantageous embodiment of the technology herein relates to breathing apparatus such as might be employed in activity underwater (e.g., diving gear) or in “outer” space (e.g., at high altitude, in orbit, or beyond the Earth's atmosphere), or in any oxygen constrained situation. Still more particularly, the technology herein relates to a quantum independent breathing apparatus (“QUIBA”) that supplies a breathable atmosphere containing oxygen, together possibly with other gases and materials, using at least one Nagel Quantum Effect Device(s) (“NQED(s)”) following the means and methods taught in US patent applications by Christopher Nagel identified below.

### BACKGROUND

Typically in situations where persons must operate in an inhospitable atmosphere—which is unbreathable, toxic, oxygen constrained, or (as in “outer” space) non-existent—they are compelled to bring their own breathable supply—generally in a tank. This can be cumbersome and limiting.

Using NQED(s), the technology herein assembles, or instantiates, or “quantum prints,” the fermions comprising a breathable atmosphere. This invites the prospect of longer forays into an inhospitable atmosphere or environment.

In many situations, it is desirable for the output supplied by the NQEDs to closely mirror the Earth's own natural atmosphere. In other situations, such as medical treatment, it may be desirable for the NQEDs to supply a mixture with enriched oxygen. There are many other situations in which still other mixtures might be desirable—such as, for example without limitation, a heliox or trimix blend for deep pressure diving, containing helium to combat nitrogen narcosis; or mixtures to which water vapor is provided to make breathing more comfortable; or mixtures to which carbon dioxide is provided, perhaps even in excess of normal atmospheric levels, to promote the growth of plants for use in greenhouses, hothouses, glasshouses, hydroponics, terraforming. horticulture, and/or agriculture.

A given mixture can be implemented with multiple NQEDs configured for various gases; or, depending on the combination, it may be possible to configure a single NQED to simultaneously produce multiple components.

The technology herein further supplies breathing apparatus such as might be employed in activity underwater (e.g., diving gear) or in “outer” space (e.g., at high altitude, in orbit, or beyond the Earth's atmosphere), or in any oxygen constrained situation. This includes oxygen constrained environments inside protective garments, or shells, or encapsulations, or enclosures, or vehicles designed to isolate the occupant(s) from the ambient external environment (which is typically presumed to be dangerous or threatening, or inhospitable in some way), including without limitation: Hazmat (“Hazardous material”) suits, “space” suits and vehicles, fire-fighting suits, bio-isolation equipment, and underwater diving equipment. This technology may also be employed in medical situations for patients who need an oxygen-rich atmosphere such as for ventilators, endotracheal tubes, bilevel positive airway pressure devices, some continuous positive airway pressure machines, or for patients with auto-immune disorders who must avoid breathing ambient microbes. Still other embodiments quantum print and deliver any substance(s) whatsoever that is consumed or taken in by or introduced into a living organism. Such embodiments can provide water or other hydration, fuel, foodstuff, nutrition, medication, drugs, pharmaceuticals, or any other substance(s) or material(s) that are needed by or helpful to the metabolism or other aspects of a living organism.

Enabling Technology of Quantum Printing Nanostructures within Carbon Nanopores—Nagel Quantum Effect Devices (NQEDS)

This application is enabled by the following six (6) US patent applications by Christopher Nagel and assigned to Quantum Elements Development, Inc., Taunton, MA (hereinafter collectively “the Nagel inventions”), each of which is incorporated herein by reference for all purposes as if expressly set forth herein, as well as Nagel's underlying technology developments (the “Nagel inventions” shall be considered a part of this specification for all purposes including but not limited to enablement of disclosure):
62/948,450, filed on Dec. 16, 2019; Ser. No.
Ser. No. 16/782,505 filed on Feb. 5, 2020; Ser. No.
Ser. No. 16/786,321 filed on Feb. 10, 2020 now 10889892;
Ser. No. 16/786,325 filed on Feb. 10, 2020 now U.S. Pat. No. 10,844,483;
Ser. No. 16/952,925 filed on Nov. 19, 2020 now U.S. Pat. No. 11,535,933; and
Ser. No. 16/808,030 filed on Mar. 3, 2020 now U.S. Pat. No. 10,988,381;
U.S. Pat. No. 10,844,483.

These Nagel applications relate to the discovery that carbon matrices can be used to produce nano-deposits, nanostructures, nanowires and nuggets comprising metals using processes including the application of electromagnetic radiation, directly and/or indirectly, to gases, nano-porous carbon, or compositions and combinations thereof, thereby pre-treating the gas, and exposing a carbon matrix to pre-treated gas in an apparatus to cause metal instantiation, nucleation, growth and/or deposition within the carbon matrix. In more detail, the Nagel inventions relate to methods of instantiating materials, such as metals and non-metals, in nanoporous carbon matrices, including processes comprising the steps of contacting a bed comprising nanoporous carbon with an activated gas while applying electromagnetic radiation to the nanoporous carbon for a time sufficient to cause instantiation, including but not limited to nucleation, growth deposition and/or agglomeration, of elemental metal or non-metal nanoparticles within and/or from carbon nanopores and nano-pore networks and matrices. The process results in nanoporous carbon compositions or matrices characterized by elemental metals and/or non-metals deposited within carbon nanopores and agglomerated elemental nanoparticles, creating elemental metal nuggets, nanowires and other macrostructures that can be easily separated from the nanoporous carbon. The processes have broad applicability in producing elemental metal composition and macrostructures and the nanoporous carbon composition can also comprise non-metal nanostructures and/or macrostructures. For example, the processes can instantiate or quantum print noble gases, such as helium, neon, argon, krypton and xenon. Additionally or alternatively, the processes can instantiate or quantum print materials containing carbon, oxygen, nitrogen, sulfur, phosphorous, selenium, hydrogen, and/or halides (e.g., F, Cl, Br and I).

Nanoporous carbon compositions further comprising metal oxides, nitrides, and sulfide such as copper oxide, molybdenum sulfide, aluminum nitride have been identified. Therefore, small inorganic molecules or compounds (e.g., molecules comprising several metal 2, 3, 4, 5, 6, 7, 8, 9 or 10 or 25 atoms) can be instantiated or printed using the processes of the Nagel invention. Examples of such small molecules include carbides, oxides, nitrides, sulfides, phosphides, halides, carbonyls, hydroxides, hydrates including water, clathrates, clathrate hydrates, and metal organic frameworks. Such devices as disclosed in the Nagel inventions or substantially similar to and/or substitutable for such devices are referred to herein as “Nagel Quantum Effect Devices (NQEDs).” Any reference herein to NQEDs, or use of the Nagel Effects, should be considered in greater generality as including any process that can instantiate, or “quantum print”, nucleate, propagate, or manifest material or matter in any way. At this present time, the means and methods of the Nagel discovery are the only known way of instantiation—however in the future, as our understanding of these processes and techniques deepens, it is possible other means and methods may be discovered, developed, or brought to our attention—and our references to “NQEDs” and “Nagel Effects” should be understood to encompass and include them as well. Thus, our references herein to “instantiation” and “quantum printing” are intended to encompass any process or technique that may employ any device, apparatus, or process that instantiates, or quantum prints, or assembles, or produces, or extracts, or isolates, or filters, or nucleates, or traps, or manifests, or supplies, or otherwise brings forth relevant materials in any way whatsoever. Some implementations may be viewed as acting on, interacting with or employing dark matter, dark energy, zero point energy, vacuum energy, or the vacuum field.

### General Notes:

All patents and patent publications cited herein are incorporated by reference.

Use of the phrase “including . . . ” should be interpreted as meaning “including but not limited to . . . ”, or “including without limitation . . . ”. Furthermore, use of the phrase “for example . . . ”, “e.g. . . . ”, or equivalent linguistic construction, should be interpreted as meaning “for example, without limitation, . . . ”.

Any reference to “computer”, “CPU”, or “processor” should be understood to include any device or means that performs computational or logical processes; such as, without limitation: computers, CPUs, GPUs, ASIC arrays, electric or electronic circuits, electronic devices, transistor devices, silicon devices, photonic devices, digital devices, analog devices, quantum devices. This should also be understood, even when used the singular sense, to include any combination of such devices and means, and regardless of the extent, if at all, to which such devices may be in communication, or networked; and regardless of the means of any such possible communication.

Any “connection” to or from a processor, or in fact any connection across which signals flow, is understood to be capable of implementation using any effective means of conveying signals—including without limitation: electrical wires, signal cables, “wireless”, radio communication, or any electromagnetic means, photonic means, optical means, mechanical means, sonic means, quantum means, or any means that uses oscillation or vibration.

### BRIEF DESCRIPTION OF THE DRAWINGS

FIG. X1 is a block diagram of an embodiment of an example system.

FIG. X2 is a block diagram of another embodiment of an example system.

FIG. X2A is a more detailed block diagram of the FIG. X1 example system.

FIG. X2B is a more detailed block diagram of the FIG. X2 example system.

FIGS. X3A, X3B, X3C and X3D are block diagrams of example non-limiting use cases.

### DETAILED DESCRIPTION OF EXAMPLE NON-LIMITING EMBODIMENTS

### General Solution/Proposition

FIG. X1 is a block diagram of an example NQED based reactor 10. In example embodiments, reactor 10 can comprise any inanimate or animate structure (open or closed) in which one or more processes such as chemical reactions take place. In some example non-limiting embodiments, reactor 10 comprises an organism such as a human, an animal or a plant, or part of such an organism or collection of such organisms in which one or more processes such as one or more chemical reactions takes place.

One or more chemical substances are supplied to reactor 10 such as via respiration or any other intake mechanism. In one embodiment, the reactor 10 consumes the one or more chemical substances to provide, enable or sustain a process or reaction. One or more reactions within reactor 10 using or based on the one or more chemical substances may in some cases produce energy such as heat, mechanical energy, or other. Other processes or reactions do not produce energy but may have other effects, e.g., effecting a change in one or more metabolic processes, organ function including but not limited to brain function or the function of any other organ, cell, system of the body, or the like. Reactor 10 may also or alternatively produce one or more reaction byproducts such as a gas, a liquid, a solid and/or other forms of matter.

As used herein, a chemical substance is not particularly limited and includes without limitation any atoms, molecules, or compounds in any form, such as in a pure form or in a mixture. Chemical substances herein include those that are reactive by themselves or towards another chemical substance, which may be referred to herein as a chemical reactant or simply reactant, and those that are inert under a given reaction condition. In some embodiments, one or more of the chemical substance(s) function as “fuel”, which generally refers to any atom, molecule, or compound that can produce energy by itself, upon reacting with another chemical substance such as an oxidizing agent, or by any other means. In some embodiments, one or more of the chemical substance(s) can be an oxidant such as oxygen gas or a mixture including oxygen gas that is used by the reactor 10 to metabolize or otherwise react other substances. Such example substances or materials that may be produced may for example comprise the elements Oxygen, Carbon, Hydrogen, Nitrogen, Calcium, Phosphorus, Potassium, Sulfur, Sodium, Chlorine, Magnesium, Iron, Fluorine, Zinc, Silicon, Gallium, Rubidium, Strontium, Bromine, Lead, Copper, Aluminum, Cadmium, Cerium, Barium, Tin, Iodine, Titanium, Boron, Selenium, Nickel, Chromium, Manganese, Arsenic, Lithium, Mercury, Cesium, Molybdenum, Germanium, Cobalt, Ruthenium, Antimony, Silver, Niobium, Zirconium, Lanthanum, Tellurium, Yittrium, Bismuth, Thallium, Indium, Gold, Scandium, Tantalum, Vanadium, Thorium, Uranium, Samarium, Tungsten, Beryllium, Radium and Lutetium or any combination, molecule or compound of such elements. Materials can comprise for example oxygen gas, water, organic compounds, inorganic compounds, lipids, proteins, carbohydrates, nucleic acids, nutrients, drugs and/or medications.

In one embodiment, at least one chemical substance is supplied by means, methods and/or processes disclosed by “the Nagel inventions”. As described above, equipment that implements such means, methods, or processes shall be referred to as “Nagel Quantum Effects Device” (“NQED”). In particular, an NQED 12 shown in FIG. X1 produces a chemical substance that may be supplied to reactor 10. NQED 12 may supply the chemical substance to reactor 10 alone or in combination with other devices such as one or more additional NQEDs or any other device. If additional NQEDs are present, the additional NQEDs may produce the same chemical substance or different chemical substances.

FIG. X2 shows plural NQEDs 12, 14 producing plural different chemical substances (e.g., reactant A and reactant B, respectively) to be supplied to reactor 10. Any number of NQEDs may produce any number of chemical substances. In more detail, each Nagel Quantum Effects Device (“NQED”) 10, 12 employs the Nagel Inventions to instantiate, or quantum print, the relevant or needed material. As one particular example, NQED 10 may produce a first chemical substance and NQED 14 produces a second chemical substance such as an oxidizing agent (e.g., oxygen (O₂)). Other embodiments may employ any other device, apparatus, or process that instantiates, or quantum prints, or assembles, or produces, or extracts, or isolates, or filters, or nucleates, or traps, or manifests, or supplies, or otherwise brings forth relevant materials in any way whatsoever. Some implementations may be viewed as acting on, interacting with or employing dark matter, dark energy, zero point energy, vacuum energy, or the vacuum field.

Advanced NQEDs 10, 12 are capable of assembling (“instantiating” or “quantum printing”) a desired chemical substance(s) or mixture of chemical substances—including but not limited to simple mono-elemental atoms and molecules (e.g., alkali metals such as Na, alkaline earth metals such as Ca, H₂, O₂, halogen molecules such as Cl₂, etc.), simple multi-elemental molecules comprising at least two elements (e.g., CO, NH₃ or H₂O₂, etc.), or complex multi-elemental molecules comprising at least two elements in various distinguished configurations (e.g., hydrocarbons, carbohydrates, alcohols, etc.).

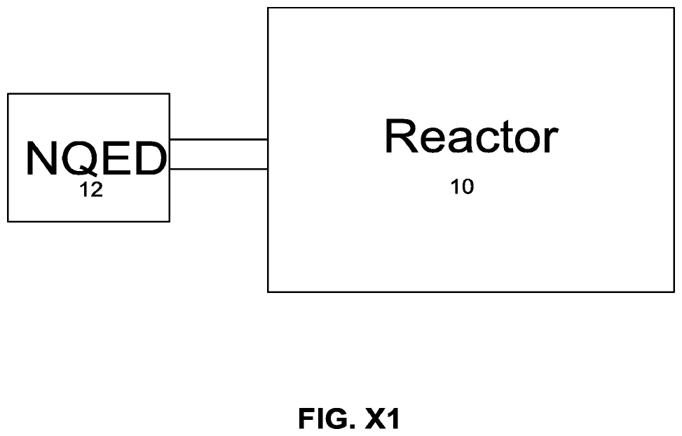

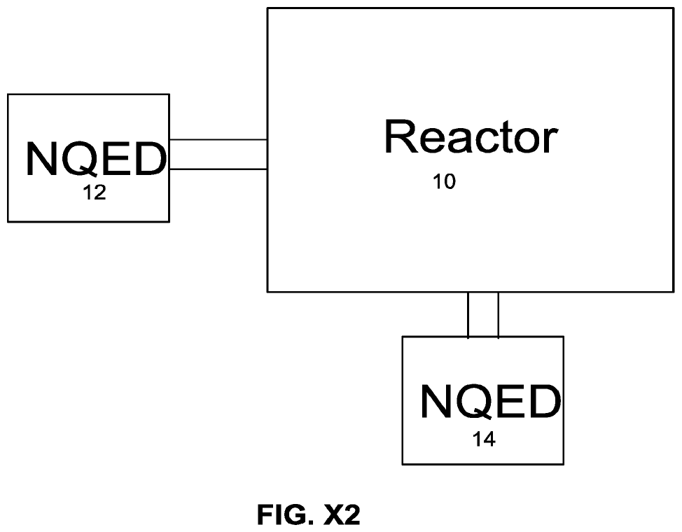

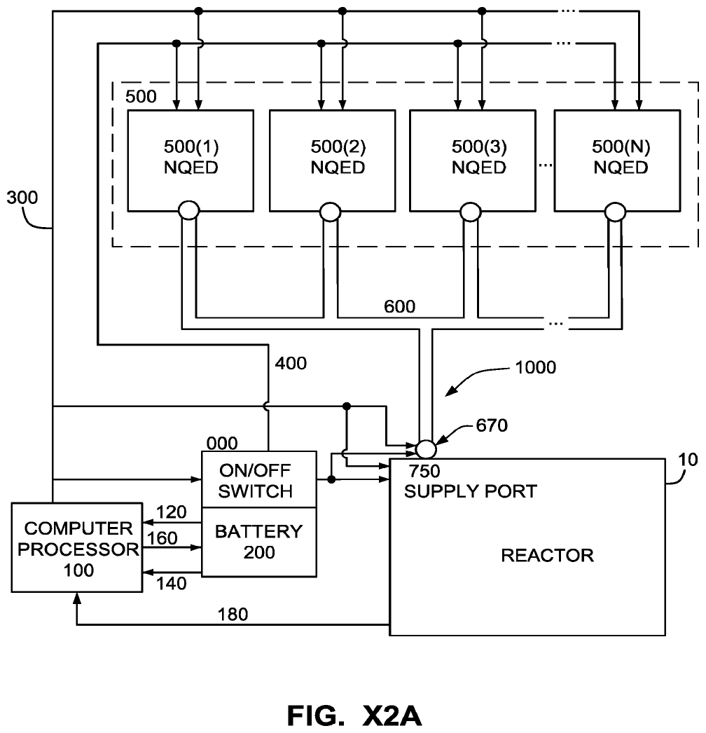

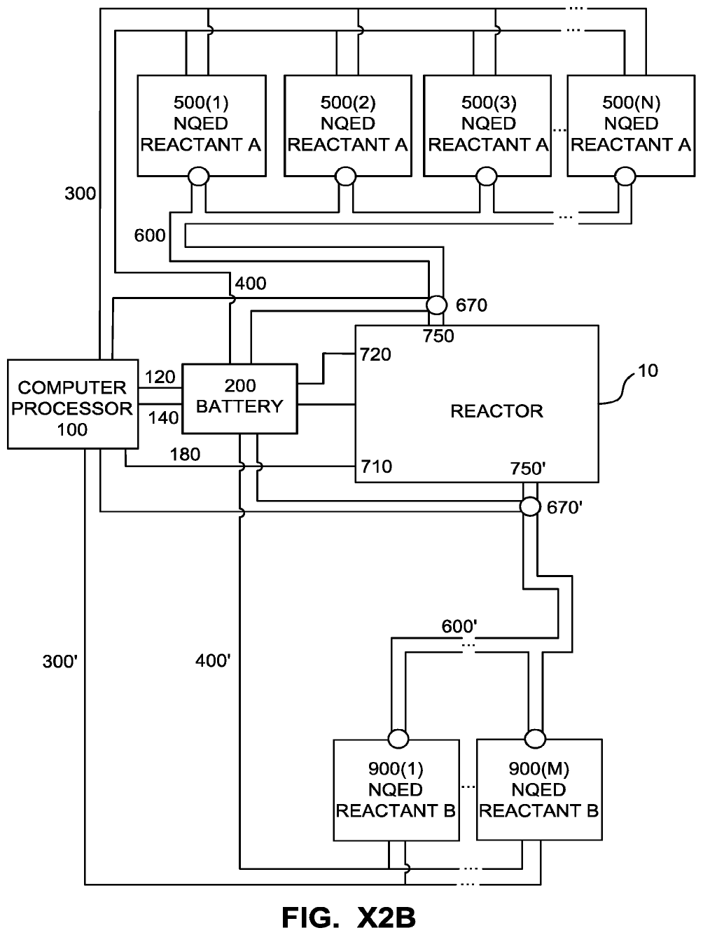

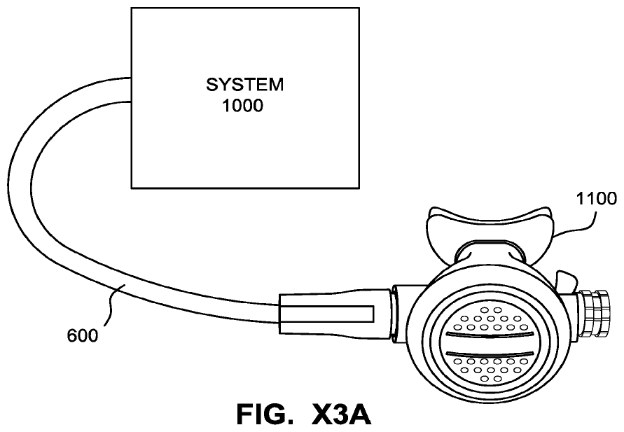

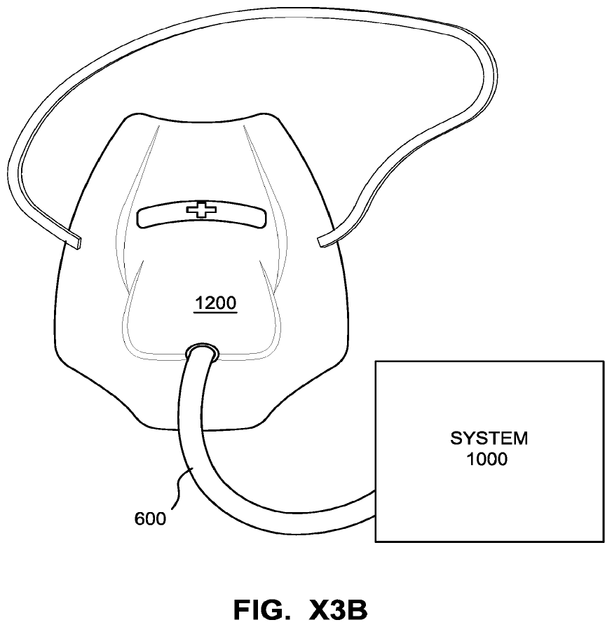

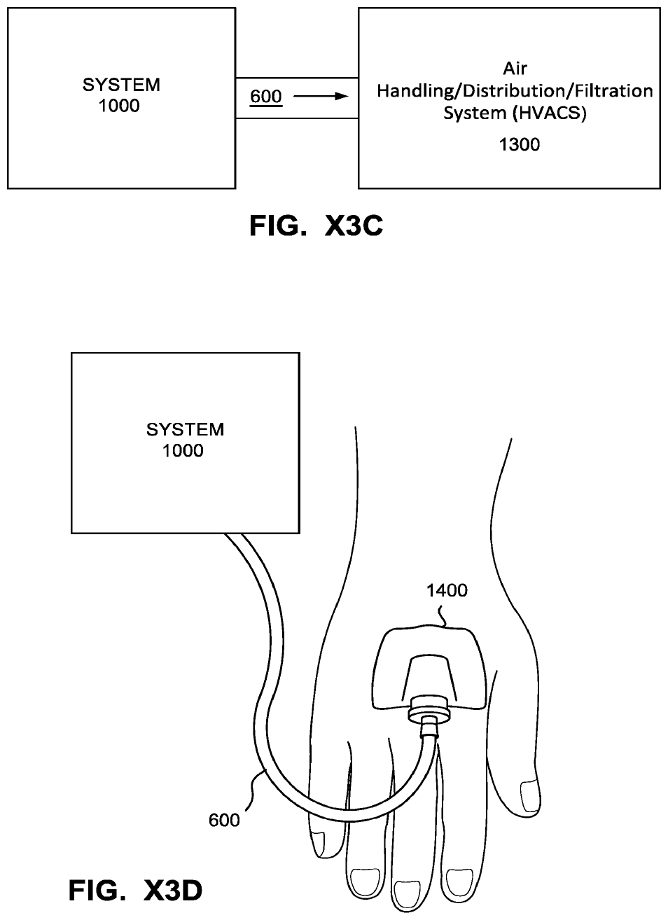

As shown in FIGS. 1 and 2, such NQEDs 10, 12 can be coupled to an apparatus such as the reactor 10 shown that can immediately, or almost immediately, or at other timing, consume the chemical substance(s) through a chemical reaction or process. Depending on the particular embodiment, the reactor 10 acting as a “sink” could be any substance-consuming apparatus; and/or a storage facility such as a tank or other container that stores the substance; or a reactant-transformation process that uses a chemical reactant as a feedstock or precursor in the production of other chemicals or materials.

Thus, example non-limiting technology herein uses at least one NQED 12, 14 which is configured to produce a chemical substance. These various dispositions of chemical substances may be generalized by the concept of “reductant sink” or “oxidizer sink”. In particular, the output(s) of such NQED(s) 12, 14 in some embodiments is/are directed through a “conduit” to a “reductant sink” or an “oxidizer sink” which receives a reductant or oxidizer and processes it in some way. However, the present embodiments are not limited to redox reactions, and instead may comprise any interaction whatsoever involving one or more chemical substances, atoms, molecules or compounds.

The FIGS. X1 and X2 systems can include one or more chemical substance users or consumers, one or more chemical substance retainers and one or more chemical substance transformers. For example, NQEDs 10 and/or 12 can be coupled to a storage facility apparatus whereby the chemical substance(s) can be retained for use elsewhere or later; or can be moved through a conduit for other processing such as being used as a feedstock or precursor to the production of other chemicals.

### Non-Limiting Chemical Substance Processing Framework

Non-limiting exemplary embodiments below share a common framework which is outlined now, with details of some specific preferred embodiments discussed later within the context of that framework.

FIG. X2A shows an NQED 500 that supplies a chemical substance to a reactor 10 via a conduit 600. In the example shown, “N” NQED(s) 500(1), 500(2), . . . , 500(N) (where N is any positive integer) may be configured to assemble the chemical substance in sufficient quantities appropriate for the sink and deliver the chemical substance to the sink, i.e., reactor 10.

In the FIG. X2B example shown, “M” NQED(s) 900 (where M is any positive integer) may be configured to assemble a second chemical substance appropriate for the sink and deliver the chemical substance to the sink, i.e., reactor 10.

Any number of additional NQEDs or banks of NQEDS may be provided to supply any number of and quantity of chemical substances individually, alternately, simultaneously or in any desired mixtures or ratios, to reactor 10.

The chemical substances produced by NQEDs 500, 900 are supplied to reactor 10 via one or more conduits 600. Thus, as material moves between points it is said to move through a “conduit”. Depending on an implementation's design and engineering constraints, a “conduit” may vary from being a trivial, almost abstract, connection to a complicated path in which a number of operations are performed, sometimes conditionally, on the subject material. Such operations may include, for example and without limitation, being: pumped, collected, combined, combined with the output of other conduits or sources, pressurized, compressed, liquefied, solidified, stored, packaged, transported, hauled, unpackaged, repackaged, gasified, uncompressed, depressurized, filtered, gated, shunted, injected, diverted, merged, blended, dissolved, extracted, sensed, tested, humidified, dehumidified, monitored, measured, regulated, accumulated, cooled, heated, or otherwise processed. Such operations may involve the use of components including for example and without limitation: pumps, sensors, gates, shunts, injectors, valves, baffles, pipes, splitters, plumbing, relays, filters, controls, accumulators, tanks, containers, reservoirs, fans, pressurizers, humidifiers, dehumidifiers, compressors, refrigerators, blenders, mixers, vats, dissolvers, extractors, coolers, heaters, gasifiers, liquefiers, and sensors and controls for flow, humidity, concentration, temperature, volume, and pressure, as well as other sensors and controls and processing equipment. Each operation may be performed zero or more times, sometimes simultaneously, and the order in which they are performed (and whether they are appropriate or necessary) depends on a particular implementation's design, tradeoffs, and constraints. Conduits may also be used to route power and signal cables.

A conduit 600, 600′ may thus without limitation comprise a single pipe or other structure capable of conducting gas or liquid, a conveyor for conducting powders or solids, a blower system for moving powders or gas, a manifold that couples the outputs of multiple NQEDs 500 together, a mixer that mixes the outputs of multiple NQEDs together, or any other suitable structure for conveying outputs of NQEDs 500 to reactor 10.

An intake manifold in the form of conduit(s) 600 in the example shown in FIGS. 2A, 2B is provided through which a substance produced by NQED(s) 500, 900 is conducted to the reactor 10. The conduit(s) 600, 600′ may also convey substances supplied by another source(s), for example, a storage tank or other production process. Such additional source(s) could be used in some embodiments and/or under some operating conditions in addition to NQED(s) 500, 900 to provide sufficient quantities and/or flow rates and/or combinations to meet demands of the reactor 10. For example, NQED(s) 500, 900 may operate for an extended period of time to develop substances for storage in storage tanks, and reactor 10 may later consume the substances stored in the storage tanks.

Item 670/670′ represents various aspects of conduits 600/600′ that may exist and be attached to processor 100 and battery 200. In particular, as discussed above, generation and/or delivery of the chemical substances may involve various additional steps and/or structures 670/670′, including for example and without limitation, those of being: pumped, collected, combined, combined with the output of other NQEDs or conduits or sources, pressurized, compressed, liquefied, solidified, stored, packaged, transported, hauled, unpackaged, repackaged, gasified, uncompressed, depressurized, filtered, gated, shunted, injected, diverted, merged, blended, dissolved, extracted, sensed, tested, humidified, dehumidified, monitored, measured, regulated, accumulated, cooled, heated, or otherwise processed through use of components including for example without limitation: pumps, sensors, shunts, injectors, valves, baffles, pipes, splitters, plumbing, relays, filters, controls, accumulators, reservoirs, tanks, containers, fans, pressurizers, humidifiers, dehumidifiers, compressors, refrigerators, heaters, blenders, mixers, vats, dissolvers, extractors, coolers, heaters, gasifiers liquefiers, and sensors and controls for flow, concentration, temperature, humidity, volume, and pressure, as well as other sensors and controls and processing equipment. Each step may be performed zero or more times, and the order in which they are performed (and whether they are necessary) depends on a particular implementation's design, tradeoffs, and constraints.

The NQED(s) 500, 900 receive electrical power as needed, from battery 200 or other suitable electrical power supply, through connections 400, 400′. Power lines 400, 400′ are thus provided from the battery 200 to the NQED(s) 500, 900. For illustrative simplicity, while all “power” connections to or from battery 200 are shown as a single line, they are intended to reflect plural conductors through which current flows.

An example system can further comprise a computer processor 100 and an on/off switch 000. The processor 100, as well as possibly the battery 200, may be connected to the “start” switch (000) which activates the various components in response to a manual or automatically generated start event. In the example 2A shown, the “start” switch 000 activates the system 10 including without limitation, all relevant components and sub-components, as appropriate. In illustration 2B the “start” switch is not shown but may be included.

In one embodiment, aspects of each NQED 500, 900 are monitored and regulated by processor 100 through bus 300/300′, which may comprise a digital data bus in one embodiment. The various monitored aspects may include, without limitation, power, temperature, humidity, configuration, pressure, flow, concentration, and any other relevant state or parameter; together with the operation of fans, pumps, valves, reservoirs, accumulators, pressurizers, compressors and/or other devices used to support the processes shown. The processor 100 may also send signals over bus 300/300′ to control aspects of the state and operation of each NQED 500, 900 such as flow control, output rate, and any other relevant state, parameter or characteristic.

As shown in FIGS. 2A, 2B, computer processor 100 provides an electronic controller that senses, monitors, regulates and controls the various aspects of chemical substance production and usage. Processor 100 is connected as needed (140, 160, 180, 300, 300′, etc.) to other various components (000, 200, 500, 900, 670, 670′, 10) to receive sensor input signals and send control signals. Computer processor 100 may be operatively coupled to a non-transitory storage device(s) that stores executable instructions. The computer processor 100 may include a CPU(s) and/or a GPU(s) that reads instructions from the storage device and executes the instructions to perform functions and operations the instructions specify. In some embodiments, the computer processor 100 may comprise or consist of hardware such as a programmable or non-programmable gate array, an ASIC or any other suitable implementation comprising hardware and/or software. In some embodiments, processor 100 may be implemented as multiple processors not necessarily mutually connected or communicating.

Computer processor 100 receives operating power 120 from the battery 200, from which it may also receive sensory signals 140 and to which it may send control signals 160. Implementations may have connections beyond those specifically illustrated here, from computer processor 100 to other components. For example, computer processor 100 may be operatively coupled to numerous input sensors; numerous output devices such as actuators, displays and/or audio transducers; and digital communication devices such as buses, networks, a wireless or wired data transceivers, etc.

In some embodiments, battery 200 provides ancillary power to various components in addition to processor 100. Battery 200 is shown external to the reactor, although in rare cases it may be internal to the reactor, such as if the reactor is implemented as or includes a fuel cell, an alternator/generator, or possesses other electrical power generation capabilities, if present, to receive and maintain charge (e.g., in the case of a patient with a cardiac pacemaker). In some implementations, battery 200 can be supplemented or replaced by other power sources such as solar panels, fuel cells, generators, alternators, or any external power sources, etc. Implementations may have connections beyond those specifically illustrated here, from battery 200 and processor 100 to other components. Many implementations are likely to include a battery 200 at least as an initial power source. In remote locations; in situations where battery acquisition, maintenance, or replacement may be difficult; or in emergency and special situations: implementations might provide for being jump-started with manually operated, or other kinetic current sources, or with solar panels.

### Example Operation of a Preferred Exemplary Embodiment Framework:

An operator (and/or the computer processor 100) activates the system by setting the “start” switch 000 to “on”. This gates power from battery 200 to the other components as appropriate, including NQEDs 500, 900 (if present), possibly processor 100, and possibly to the chemical substance-sink for embodiments that require preparation in anticipation of substance flow. Once started, processor 100 monitors, coordinates, and regulates, as necessary, the activity and interaction of all components. The NQEDs 500, 900 (if present) are started under control of processor 100—with the appropriate environment being established—including for example without limitation: power, temperature, humidity, pressure, charge, and electromagnetic fields. This involves sensors and controls in NQEDs 500, 900 (if present) the signals of which are transmitted through bus 300/300′ to and from the processor 100.

Once ready, the NQEDs 500, 900 (if present) are operationally activated under control of processor 100, which thereafter senses, monitors and controls NQEDs 500, 900 to ensure proper operation. In active state, NQEDs 500 in one embodiment instantiates the chemical substance to be delivered, which, in the exemplary preferred embodiment, is atoms or molecules, such as oxygen (O₂) mixed with other gases or any other chemical substance. The material(s) emitted by the NQEDs 500 is/are collected by the conduit 600 which may process the material(s) in various ways (denoted by 670) as appropriate before the chemical substance is delivered to the reactor/chemical substance sink 10 through its chemical substance intake 750. Item 670 represents various aspects of conduit(s) 600 that may exist and be attached to processor 100 and battery 200. Similarly, NQEDs 900 in one embodiment instantiate a second agent which, in the exemplary preferred embodiment, are atoms or molecules, such as nitrogen (N₂) or any other chemical substance. The oxidant material(s) emitted by the NQEDs 900 is/are collected by the conduit 600′ which may process the oxidant in various ways (denoted by 670′) as appropriate before the material(s) and/or mixtures is/are delivered to the reactor 10 through its reactant intake 750′.

After a chemical substance sink operation completes—which may be determined in various ways depending on the particular specific embodiment, including being: signaled by the operator setting the “start” switch to “off”; signaled via some computer interaction or decision; signaled by the sink (such as if the sink is a storage tank which reaches a full state)—the computer 100 conducts a proper close down for the NQEDs 500, 900, conduits 600, 670, 670′, reactor 10, other sink apparatus (see e.g., examples below), battery 200, and itself 100.

Different Kinds of Reactors and Other Apparatus that Receive NQED Outputs

As discussed above, each NQED 500 may provide a chemical substance to a reactor 10 which consumes the chemical substance. The reactor 10 may thus in some embodiments be considered to be a “sink”—i.e., a device that “sinks” (takes in) the substance produced by the NQED 500. Examples of sinks include, without limitation: consumers or users, retainers, and transformers.

Similarly, each NQEDs 900 may in some embodiments provide an additional chemical substance such as an oxidizer, or other material used to react with the first substance and/or used in conjunction with the first substance. NQEDs 900 may supply such additional chemical substance to reactor 10, which consumes the chemical substance such as oxidizer or other material. Such reactor 10 may thus in some embodiments be considered an “oxidizer sink” because it consumes the oxidizer. Examples of oxidizer sinks include, without limitation: oxidizer consumers or users, oxidizer retainers, and oxidizer transformers. Many reactions serve as both chemical substance consumers and oxidizer consumers.

### Example Reductants for Redox Reactions

Not all reactions or chemical effects that take place within an organism are redox reactions, but some may be. In some embodiments, the NQEDs 500, 900 can produce chemical substances such as reductants or other chemical substances including, but not limited to, the many and varied substances containing hydrogen, carbon, nitrogen, oxygen, calcium, sodium, potassium, phosphorus, sulfur, or other materials, such as other oxidizable materials, such as, by way of example but not limited to:
hydrogen (H₂)
carbon (C)
carbon monoxide (CO);
ammonia (NH₃);
   hydrocarbons, such as:
alkynes [e.g., hydrocarbons having a formula of (CₙH₂ₙ₋₂), n is 2 or greater, such as ethyne (C₂H₂), etc.];
cycloalkanes [e.g., those having one ring, which can have a formula of (CₙH₂ₙ), n is 3 or greater];
alkenes [e.g., hydrocarbons having one double bond with a formula of (CₙH₂ₙ), n is 2 or greater]
alkanes (paraffins)[e.g., those having a formula of (CₙH₂ₙ₊₂) including by example, methane (CH₄), ethane (C₂H₆, CH₃CH₃), propane (C₃H₈), butane (C₄H₁₀); pentane (C₅H₁₂) through octane (C₈H₁₈)—the gasoline predistillates; nonane (C₉H₂₀) through hexadecane (C₁₆H₃₄);
aromatic hydrocarbons (arenes) such as benzene, substituted benzenes such as toluene, xylene, etc. or bicyclic or polycyclic aryls];
   and a vast collection of other organic compounds, of which a small sample includes:
alcohols, such as alkanols [such as monohydric (CₙH₂ₙ₊₁OH), diols or polyols] unsaturated aliphatic, alicycllic, and other alcohols having various hydroxyl attachments];
nitroalkanes such as nitromethane (CH₃NO₂);
carbohydrates.
medications
pharmaceuticals of any type
nutritional agents of any type
any other substance that can be used by or useful in or with one or more metabolic processes.

In many cases, NQEDs 500, 900 can directly instantiate or quantum print the chemical substance, the production of which might otherwise require transformation by a chemical reaction or a different source.

### Example Oxidants (Oxidizing Agents)

NQEDs 500, 900 can instantiate (or “quantum print”) oxidants (alternatively referred to herein as oxidizers or oxidizing agents or oxygen-based chemical substances) including without limitation:
oxygen (O₂), O₃, O₄, and O₈, etc.
H₂O₂
H₂O, which exothermically oxidizes alkali metals, alkaline earth metals, etc. and can exothermically react with alkali metal oxides or alkaline earth metal oxides, such as CaO;
halogen molecules, such as F₂, Cl₂, Br₂, etc. and other reactive metals (e.g., metal oxides) or non-metals.

### Example Consumers or Users

Reactors 10 that are consumers include, but are not limited to, apparati, devices, systems and means that immediately, or almost immediately, or at a later time consume or use chemical substance through metabolic or other chemical reaction(s). Chemical substance consumers may also often comprise oxidizer consumers.

Example Chemical substance or Oxidizer Retainers

Reactors 10 that are chemical substance/reductant and oxidizer retainers include apparati and means that store one or more chemical substances such as oxidizer for use elsewhere or later. This includes for example:
tanks or bottles (liquid or gasses), caves (gasses), bags (solid), conduits, or any other vessel or other structure that at least for a moment or an instant (or for any short or long time period), either while in transit or statically, stores a quantity of chemical substance/reductant or oxidizer.

### Example Chemical Substance Transformers

Reactors 10 that are transformers for one or more chemical substances such as chemical substances include apparati and means that extract and convert from one or more chemical feedstock substances such as chemical substances/reductant, such as hydrocarbons, which can for example without limitation be processed using chemical reactions such as substitution and addition of other reagents such as chlorine, or other chemicals; and/or physical processes such as mixing, blending, melting, softening, refining, hardening, vaporizing, cooling, distilling, liquefying, solidifying, freezing, crushing, powdering, exuding, extruding, rolling, smelting, alloying to produce more advanced products and including:
solvents (nail polish, paints, naptha (mothballs));
lubricating oils;
paraffin wax;
asphalt;
synthetics, such as polyester;
polymers, such as polyethylene, polypropylene, polystyrene, acrylates;
benzene, toluene, xylene isomers;
plastics;
fertilizers;
pesticides.

### Example 1 Embodiments/Use Case

The FIG. X2B example non-limiting embodiment described above may be used to implement a quantum independent breathing apparatus (“QUIBA”) system 20, where pictorial elements reflect implementations in which a Nagel Quantum Effect Device (“NQED”) assembly employs [Nagel] quantum effects to catalytically instantiate/filter/isolate/extract/nucleate/“quantum print” ambient fermions and assemble them into oxygen, possibly together with other breathable material, including without limitation: nitrogen, helium, argon, and/or water vapor. In such case, the reactor 10 comprises a human body, which takes in the output of the NQEDs 500, 900 through respiration (breathing, inhaling, etc.)

As described above, the FIG. X2B example system 20 includes a computer processor or other system controller 100; a battery or other electrical power source 200; and a delivery supply port 700. In some example embodiments, the port 700 may be: a “scuba-like” regulator-type mouthpiece breathing gear 1100 (for the personal portable QUIBA implementations such as diving gear, hazmat suits, fire-fighting gear) such as shown in FIG. X3A; in other preferred embodiments, port 700 may be input to a ventilation or environmental control system 1300 (see FIG. X3C) for vehicular or stationary implementations (submarines, high altitude aircraft, and space craft); input to a hospital “oxygen tent” or oxygen mask 1200 as shown in FIG. X3B for some medical implementations; and input to other medical equipment such as ventilators, endotracheal tubes, bilevel positive airway pressure devices, nasal cannula, some continuous positive airway pressure machines, a hospital central oxygen supply for connection to oxygen masks and the like, or other oxygen supply arrangements. Depending on the embodiment, port 700 may include a number of components or subsystems such as without limitation some or all of the following:
face, mouth, or nose pieces (see FIGS. X3A, X3B for examples);
tubing, fans, ducts, blowers, compressors, valves, plumbing, louvres;
conventional venting or exhaust components such as vents, exhaust valves or ports (e.g., to vent CO2 and H2O produced when a subject exhales);
air conditioning, environmental control systems, ventilation, circulation, heating, humidifying, dehumidifying and cooling apparatus (see FIG. X3C).

One example preferred embodiment represents personal portable wearable scuba-like gear 1100 (see FIG. X3A) where the supplied atmosphere is approximately 22% oxygen, 78% nitrogen and port 700 is scuba-like face, nose, and mouth gear 1100. One example preferred embodiment represents personal portable wearable scuba-like gear where the supplied atmosphere is a “heliox” mixture of oxygen and helium. For example, such mixture can be 21% oxygen and 79% helium. Another example “helitrox” or “trimix” mixture is 26% oxygen, 17% helium, and 57% nitrogen. Helium is often substituted for nitrogen in deep sea diving to reduce the chance of decompression sickness.

In one example preferred embodiment, for a submarine, an aircraft, a space craft, a building such as a home, a business or a warehouse, a train, or any other inhabitable structure, the supplied atmospheric production is larger scale and the port 700 is a ventilation system 1300 as shown in FIG. X3C.

In one example preferred embodiment, for medical treatment, the supplied atmosphere has enriched oxygen (exceeding 22% such as exceeding 25% or exceeding 30% or exceeding 50% or exceeding 75% or exceeding 90% or substantially at 100% pure oxygen) and the port 700 is an “oxygen tent” or oxygen mask 1200 as shown in Figure X3B. In one embodiment as shown in FIG. X3B, an oxygen mask 1200 or other device or arrangement communicates with the mouth of one or more human subjects. In such arrangements, the oxygen can be mixed with an inert or substantially inert gas such as nitrogen, helium, argon, neon or other breathable gas. The pressure (i.e., quantity per volume) of gas may also be controlled. Additional devices may be employed to filter the gas and/or control or adjust the temperature of the gas.

In addition, in some embodiments, the same conduit 600 used to deliver a breathing gas to the organism is also used by the organism to exhale. Such exhalation often is 4% to 5% by volume of carbon dioxide and also typically includes water vapor. In some arrangements such as the regulator mouthpiece 1100 of FIG. X3A, the exhaled gas is vented to the environment. In other arrangements such as the oxygen mask 1200 shown in FIG. X3B, system 1000 may also be equipped with a water vapor condenser to remove water vapor from the exhaled gas and/or may be equipped with a vent to vent exhaled gas to prevent recirculation of high levels of carbon dioxide or other exhaled byproducts of metabolic processes. In many preferred embodiments, a slight amount of humidity (e.g., water vapor) may be added to the supplied atmosphere to reduce the drying effect of the mixture.

In one example preferred embodiment, for medical treatment, the supplied atmosphere has enriched oxygen (exceeding 22%) and the port 700 is a nasal cannula or oxygen mask.

In another example preferred embodiment, the reactor 10 comprises one or plants within a greenhouse (e.g., hothouse, glasshouse) and agriculture, and the supplied atmosphere contains nitrogen and is enriched with carbon dioxide up to 1500 ppm or more (far exceeding the normal 400 ppm atmospheric average) which can increase plant yields by as much as 30%.

In some embodiments, the amount of oxygen may be depressed relative to typical atmospheric gas. For example, one embodiment produces a gas mixture of 10% oxygen, 70% helium, and 20% nitrogen.

As described above, in example embodiments a computer processor 100 provides an electronic controller that senses, monitors, regulates and controls the various other aspects of the implementation, and is connected as needed to other various components to receive sensor input signals and send control signals. In this embodiment, sensors can for example be used to detect the oxygen and other gas content of the breathing gas, the temperature of the breathing gas, the humidity of the breathing gas, the flow of the breathing gas, and other parameters of the breathing gas. Computer processor 100 may be operatively coupled to a non-transitory storage device that stores executable instructions.

The computer processor 100 may include a CPU(s) and/or a GPU(s) that reads instructions from the storage device and executes the instructions to perform functions and operations the instructions specify. The computer processor 100 may in some embodiments include or comprise logic arrays such as programmable gate arrays, ASICs or other hardware. Some of the illustrated CPU connections may be unnecessary in certain embodiments.

In the FIG. X2B example shown, the “n” NQED(s) 500 may be configured to assemble oxygen and nitrogen in the proportion 22% to 78%—mirroring Earth's atmosphere.

These “n” NQED(s) 500 receive electrical power as needed, from battery 200 through lines 400. Aspects of each NQED 500 are monitored and regulated by processor 100 through connections 300. The various monitored aspects may include, without limitation, power, temperature, humidity, configuration, flow, pressure and the operation of fans, pumps, valves, reservoirs, accumulators, tanks, compressors. The processor 100 may also control aspects of the operation of each NQED 500 such as flow control, output rate, pressure, etc.

A conduit(s) 600 in the example shown is provided through which the oxygen, nitrogen, and other atmospheric material assembled by NQED(s) 500 is conducted to portal 700 where they are made available to be inhaled. The conduit(s) 600 may also convey materials supplied by another source(s), for example, a storage tank. In many preferred implementations, the conduit(s) may include equipment (illustrated by 670)—including a compressor and at least one backup storage tank together with plumbing, valves, sensors, controls, and pumps to ensure the backup tank is normally kept filled with compressed gases. This is done as a failsafe measure. In event the NQED or other equipment fails for any reason, the backup tank can be manually or automatically opened (independently of the other equipment) and connected to port 700, or accessed directly by the user. The backup tank should have sufficient supply to allow the user to reach safety or take definitive remedial action.

Item 670 represents various aspects of conduits 600 that may exist and be attached to processor 100 and battery 200. Generation and/or delivery of the breathable gas(es) may involve various steps and associated structures including, without limitation, being: pumped, gated, diverted, monitored, measured, regulated, collected, accumulated, stored, combined with the output of other NQEDs, pressurized, compressed, cooled, heated, liquefied, humidified, dehumidified, filtered, vented or otherwise processed; and various components including without limitation: pumps, sensors, valves, relays, controls, accumulators, reservoirs, tanks, fans, compressors, refrigerators, heaters, liquefiers, humidifiers, dehumidifiers, filters, vents and sensors and controls for flow, temperature, humidity, volume and pressure, as well as other sensors and controls and processing equipment. These steps and components may occur zero or more times and occur in any order.

In the example shown, each Nagel Quantum Effect Device (“NQED”) 500 employs [Nagel] quantum effects to catalytically instantiate or “quantum print”/filter/isolate/extract/nucleate ambient fermions and assemble them into various materials using the means and methods taught or disclosed in the above-referenced Nagel patent application. Other embodiments may employ any other device, apparatus, or process that assembles, instantiates, “quantum prints”, produces, extracts, isolates, filters, nucleates, manifests, or otherwise brings forth fermions or clusters of fermions in any way whatsoever. One or more of NQED(s) 500 may instantiate oxygen. In some embodiments, one or more additional NQED(s) 500 may instantiate one or more other breathable gas(es) such as nitrogen, helium, argon, etc. A conduit 600 may provide a chamber that mixes the gas(es) produced by the various NQED(s) 500 to provide a breathable gas mixture. Computer processor 100 can, based on software and/or hardware processing, control the active number of and/or production rate of different NQED(s) 500 to produce a desired flow rate and/or desired pressure and/or desired mixture ratio of gas(es). The computer processor 100 can change the flow rate and/or pressure and/or mixture ratio depending on sensed environmental or other conditions, specified user preference, different dive phases for deep sea diving, and different activity levels for human subjects (e.g., exercise, sleep, etc.)

As the atmosphere is assembled and emitted by the, at least one, NQED(s) 500, it is conducted to the delivery port 700 by the conduit(s). Depending on the implementation and engineering constraints, generation and/or delivery may involve various steps and associated structures including, without limitation, being: pumped, gated, diverted, monitored, regulated, collected, pressurized, accumulated, stored, combined with the output of other NQEDs, compressed, cooled, heated, liquefied, humidified, dehumidified, enriched, or otherwise processed; and various components including without limitation: pumps, sensors, valves, relays, controls, accumulators, reservoirs, tanks, fans, pressurizers, compressors, refrigerators, heaters, liquefiers, humidifiers, dehumidifiers and sensors and controls for flow, temperature, humidity, volume and pressure, as well as other sensors and controls and processing equipment. These steps and components may occur zero or more times and may occur in any order depending on an implementation's design, tradeoffs, and constraints.

The supplied atmosphere is directed to the delivery port where it is consumed by the organism. This may be implemented in a variety of ways depending on the design.

In any of these types of implementations can also use additional NQEDs 500 to assemble/provide additional atmospheric ingredients into the delivered mixture. For many implementations this is the standard mix of 22% oxygen with 78% nitrogen. Other breathable mixtures may be less (e.g., 10%) oxygen or more (e.g., 100%) oxygen.

Breathing 100% oxygen at standard pressures can be harmful for humans, but breathing 100% oxygen may be acceptable at reduced pressure.

Example Non-Limiting Operation of the QUIBA: Implementation benefits from attention to issues that may require use of ancillary power, including without limitation:
creating the conditions necessary to support the Nagel Effect;
activating the Nagel Effect once the prerequisite conditions are established;
sustaining, to the extent necessary, the Nagel Effect once activated;
circulating, compressing, and pumping the gases as necessary, and implementing other functions contemplated for conduits 600;
managing the user's exhalation as needed;
handling cooling and heating issues as required.

Aside from the first three points which relate specifically to the Nagel Quantum Effect Services, these issues are well studied and can be resolved by a person skilled in the art of breathing apparatus design. Possible solutions include using line current, batteries, or outside sources to start or to operate the system.

Similar techniques can also be applied to the first three issues.

Although the Nagel Effect is not yet fully understood, it appears, that once started, it will continue assembling fermions with little or no additional ongoing power requirement as long as the proper operating environment is maintained. While the power required to start a NQED seems modest in many implementations, its correlation with a Device's performance has not been clearly determined.

### Example Use Case 2:

FIG. X3D is a block diagram of an additional embodiment in which the chemical substance produced by system 1000 is supplied to the reactor/organism 10 other than through breathing. In the particular example shown in FIG. X3D, the conduit 600 delivers a liquid such as a saline solution with one or more added chemical substance(s) that can be injected into the circulatory system (blood vessel) of an organism. In such embodiments, system 1400 may include a needle that is used to penetrate the wall of a blood vessel so that a chemical substance can be injected directly into the blood vessel. In such embodiments, system 1000 is further controlled using conventional technology to regulate the pressure, salinity and concentration of the liquid mixture injected into the organism.

Although FIG. X3D shows injection into the bloodstream as a non-limiting example, other ways and methods of providing the chemical substance(s) generated by NQEDs 500, 900 may be used instead. Such methods may include for example injection, subcutaneous injection, application to the surface of the skin (transdermal), oral/swallowing, pulmonary, buccal, ocular, nasal, sublingual, vaginal, anal, or by any other method of delivering a chemical substance to the organism. Chemical substances provided by NQEDs 500, 900 to the organism can include depressants that slow down nerve and brain activity, hallucinogens that alter what we see and hear, painkillers that block nerve impulses, performance enhancers that improve muscle development, stimulants that increase nerve and brain activity, and any other substance(s) (controlled or uncontrolled) that provides nutrition, improved wellness, therapeutics, treatment, or any other effect on an organism including but not limited to a human.

While the invention has been described in connection with what is presently considered to be the most practical and preferred embodiments, it is to be understood that the invention is not to be limited to the disclosed embodiments, but on the contrary, is intended to cover various modifications and equivalent arrangements included within the spirit and scope of the appended claims.

1. A breathing apparatus comprising:
at least one NQED arrangement to assemble, instantiate, or “quantum print” breathable gas(es); and
at least one supply port operatively coupled to the at least one NQED arrangement to deliver said breathable gas(es).

2. The breathing apparatus of claim 1 wherein the breathable gas(es) comprises oxygen.

3. The breathing apparatus of claim 1 wherein the NQED arrangement assembles breathable gas(es) containing oxygen.

4. The breathing apparatus of claim 1 wherein the NQED arrangement comprises a plurality of NQEDs.

5. The breathing apparatus of claim 1 wherein the NQED arrangement assembles breathable gases containing approximately 22% oxygen and 78% nitrogen.

6. The breathing apparatus of claim 1 wherein the NQED arrangement assembles breathable gases containing oxygen in a concentration exceeding 22%;

7. The breathing apparatus of claim 1 wherein the NQED arrangement assembles breathable gases containing a mixture of oxygen and helium.

8. The breathing apparatus of claim 1 wherein the delivery port comprises scuba-like gear comprising at least one of a mouthpiece, a nose piece, or a face mask.

9. The breathing apparatus of claim 1 wherein the delivery port comprises a nasal cannula

10. The breathing apparatus of claim 1 wherein the delivery port comprises a medical oxygen tent.

11. The breathing apparatus of claim 1 wherein the delivery port comprises a ventilation system.

12. The breathing apparatus of claim 1 wherein the delivery port comprises a ventilation system in a submarine.

13. The breathing apparatus of claim 1 wherein the delivery port comprises a ventilation system in a spacecraft.

14. The breathing apparatus of claim 1 wherein the delivery port lies within a hazmat suit.

15. The breathing apparatus of claim 1 wherein the delivery port lies within a space suit.

16. The breathing apparatus of claim 1 wherein the supplied atmosphere contains water vapor.

17. The breathing apparatus of claim 1 further comprising a battery applying power to the at least one NQED arrangement.

18. The breathing apparatus of claim 1 further comprising a fuel cell applying power to the at least one NQED arrangement.

19. The breathing apparatus of claim 1 further comprising a generator applying power to the at least one NQED arrangement.

20. The breathing apparatus of claim 1 further comprising a line current connection for connecting power to the at least one NQED arrangement.

21. The breathing apparatus of claim 1 wherein the NQED continuously assembles fermions with little or no additional ongoing power requirement as long as the proper operating environment is maintained.

22. The breathing apparatus of claim 1 further comprising an electronic controller connected to control the at least one NQED arrangement.

23. The breathing apparatus of claim 22 further including a start switch connected to the electronic controller.

24. The breathing apparatus of claim 1 wherein the NQED arrangement assembles gases including a mixture of nitrogen and carbon dioxide.

25. The breathing apparatus of claim 1 wherein the delivery port vents into a greenhouse, hothouse or glasshouse.

26. A substance delivery system for delivering a substance to an organism comprising:
at least one NQED arrangement to assemble, instantiate, or “quantum print” at least one chemical substance; and
at least one supply port operatively coupled to the at least one NQED arrangement to deliver said chemical substance to the organism.

27. The substance delivery system of claim 26 wherein the supply port delivers the chemical substance to the organism by injection, subcutaneous injection, application to the surface of the skin (transdermal), oral/swallowing, pulmonary, buccal, ocular, nasal, sublingual, vaginal, and/or anal.
